(19) United States
(12) Patent Application Publication
(10) Pub. No.: US 2024/0366104 A1
(43) Pub. Date: Nov. 7, 2024

(54) WIRELESS, CONTINUOUS MONITORING OF DAILY STRESS AND MANAGEMENT PRACTICE VIA SOFT BIOELECTRONICS

(71) Applicant: Georgia Tech Research Corporation, Atlanta, GA (US)

(72) Inventor: Woon-Hong Yeo, Atlanta, GA (US)

(21) Appl. No.: 18/681,651

(22) PCT Filed: Sep. 29, 2022
(86) PCT No.: PCT/US22/45129

§ 371 Date: Feb. 6, 2024

Related U.S. Application Data
(60) Provisional application No. 63/249,845, filed on Sep. 29, 2021.

(51) Int. Cl. A61B 5/0533; A61B 5/00; A61B 5/01; A61B 5/16; H01L 21/768

(52) U.S. Cl. CPC A61B 5/0533; A61B 5/01; A61B 5/165; A61B 5/6833; A61B 2562/0271; A61B 2562/125; A61B 2562/164; H01L 21/768

(57) ABSTRACT

A physiological monitoring device (200) for application to a user's skin (10) employs a clinical-grade medical film (202) with a skin-compatible adhesive (402) disposed on the bottom side (201). Electrodes (420) are disposed on the bottom side (201) and sense a physiological metric from the user's skin (10). An elastomeric membrane (440) is integrated with the top side of the clinical-grade medical film (202). Undulated wires (430) are on an elastomeric membrane. An electronic circuit (204) is disposed on the elastomeric membrane (440) and is electrically coupled to the electrodes (420) via the undulated wires (430). The electronic circuit (204) senses the physiological metric from the electrodes (420), converts the physiological metric to a digital value, and stores the digital value for communication of the digital value to a remote device (210).

### CROSS-REFERENCE TO RELATED APPLICATION(S)

This application claims the benefit of U.S. Provisional Patent Application Ser. No. 63/249,845, filed Sep. 29, 2021, the entirety of which is hereby incorporated herein by reference.

### STATEMENT OF GOVERNMENT SUPPORT

This invention was made with government support under grant number 2024742, awarded by the National Science Foundation. The government has certain rights in the invention.

### BACKGROUND OF THE INVENTION

### 1. Field of the Invention

The present invention relates to personal metric sensing devices and, more specifically, to a wearable personal metric sensing device.

### 2. Description of the Related Art

Work-related stress and its adverse outcomes have resulted in severe risks to individuals and puts a burden on public health. For example, it is estimated that excessive workplace stress causes 120,000 deaths with $190 billion in healthcare costs per year in the United States. Individuals with high levels of stress suffer from negative physical and psychological consequences, such as sleeping problems, depression, and cardiovascular disease.

Early detection and management of excessive stress are critical to maintaining a healthy life. Stress levels are typically estimated by self-graded response questionnaires or behavior data studies, which can be subjective and hard to quantify. Recently, to evaluate the effectiveness of stress management, wearable systems have been developed by measuring physiological signals from the heart, muscle, and brain. Electrodermal activity, which is also referred to as galvanic skin response (GSR), is one of the key indicators that directly evaluate stress arousal and cognitive states by sensing the sympathetic nervous system.

Cognitive and emotional stressors activate the sympathetic nervous system (SNS), promoting the eccrine glands' secretion to generate sweat on the skin. GSR sensors can monitor sympathetic activity by detecting variation of the ionic permeability of sweat gland membranes. The phasic signal of GSR, which exhibits a rapid time-varying response, can be correlated with arousals by SNS. Thus, identification of the phasic component of GSR allows real time quantification of stress. GSR is typically measured by attaching wired gel-electrodes on an individual's fingers or hands where sweat glands are dense. However, sensing GSR from these locations result in significant motion artifacts and data loss from frequently disconnected wires. Recent advances in miniaturized wearable devices allow wireless detection of GSR's on other areas, such as wrists and arms. These devices, however, still suffer from side effects, including substantial motion artifacts caused by rigid sensor dissociation from the skin, skin irritation from electrolyte gels, and discomfort due to aggressive fixtures and straps.

Recent reports indicate that wearable devices can measure stress intervention and the effectiveness of the management practice. Nevertheless, existing devices still rely on bulky, rigid sensors and electronics, limiting the continuous and long-term use in daily life. In addition, all of those studies have been conducted in controlled laboratory environments with limited monitoring time, less than an hour. Effective stress management requires real time measurement during normal daily activities.

Therefore, there is a need for a comfortable and wearable GSR sensor that can be worn during the course of a user's daily activities.

### SUMMARY OF THE INVENTION

The disadvantages of the prior art are overcome by the present invention which, in one aspect, is a physiological monitoring device for application to a user's skin that employs a clinical-grade medical film having a top side and an opposite bottom side with a skin-compatible adhesive disposed on the bottom side. At least two electrodes are disposed on the bottom side and are configured to sense at least one physiological metric from the user's skin. An elastomeric membrane is integrated with the top side of the clinical-grade medical film. A plurality of undulated wires is printed on an elastomeric membrane. An electronic circuit is disposed on the elastomeric membrane and is electrically coupled to the at least two electrodes via the plurality of undulated wires. The electronic circuit includes a plurality of circuit elements that sense the at least one physiological metric from the at least two electrodes, that converts the at least one physiological metric to a digital value, and that stores the digital value for communication of the digital value to a remote device.

In another aspect, the invention is a method of making a skin-mountable electronic circuit, in which at least two electrodes are deposited onto a clinical-grade medical film having a bottom side with a skin-compatible adhesive so that the at least two electrodes are exposed from the bottom side. A plurality of undulated circuit interconnects is generated on an elastomeric membrane. A plurality of circuit elements is deposited onto the undulated circuit interconnects. The elastomeric membrane is integrated with the clinical-grade medical film so that the electrodes are in electrical communication with the circuit elements. The elastomeric membrane, the undulated circuit interconnects and the circuit elements enveloped with an elastomer.

In yet another aspect, the invention is a method of monitoring a stress level of a user, in which a circuit for sensing at least one physiological metric that is mounted on a substrate that includes a flexible, stretchable and breathable material is applied to the skin of the user. The at least one physiological metric is sensed and a digital value representative thereof is stored in the circuit. The digital value representative of the at least one physiological metric is read with a remote device. A digital value representative of the at least one physiological metric is correlated to a corresponding stress level. Data indicative of the stress level is presented to the user.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a flow diagram showing one representative method of analyzing stress.

FIG. 2 is a flow diagram showing one representative method of processing stress metrics.

FIG. 3 is a schematic diagram showing a wearable stress sensor applied to a user's wrist and interacting with a remote device.

FIG. 4 is a side view schematic diagram of a wearable stress sensor applied to a user's wrist.

FIG. 5 is an exploded schematic diagram of a wearable stress sensor.

FIG. 6 is a block diagram showing electronic units employed in one embodiment of a wearable stress sensor.

FIG. 7 is a schematic diagram of a second embodiment of an electrode structure.

FIG. 8 is a flow chart showing one method of making a skin-wearable sensor system.

FIG. 9 is a series of schematic diagrams showing one method of making an electronics portion of a wearable stress sensor.

FIG. 10 is a series of schematic diagrams showing one method of making an electrode portion of a wearable stress sensor.

FIG. 11 is a schematic diagrams showing an electronics portion integrated with an electrode portion of a wearable stress sensor.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. The present disclosure should in no way be limited to the exemplary implementations and techniques illustrated in the drawings and described below. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

The present invention includes a class of technologies that include soft, nanomembrane biosensors, and stretchable bioelectronics. The all-in-one wearable device offers wireless, portable, continuous, and long-term (i.e., more than 6 hours in one embodiment) recording of galvanic skin resistance (GSR) and temperature to assess stress on the skin during normal daily activities. Also, the ultrathin and lightweight system can monitor stress management practice and the intervention efficacy via statistical analysis. The soft wearable device that makes conformal, intimate contact to the skin without electrolyte gels allows the recording of high-quality physiological data with minimized motion artifacts.

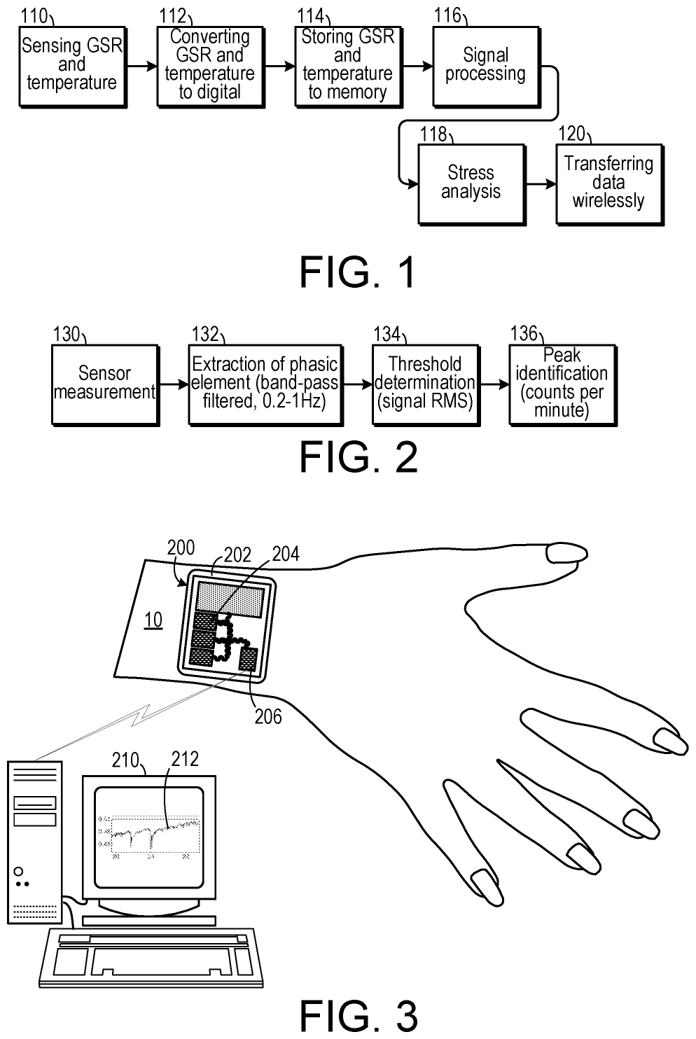

As shown in FIG. 1, in one representative embodiment, the user's skin temperature and GSR are sensed 110, converted to a digital value 112 and stored in a digital memory 114. Signal processing 116 is applied to the stored data to render stress-related metrics, which are analyzed 118 and transferred to a remote device wirelessly 120. As shown in FIG. 2, the measurements are taken by a sensor suite affixed to the user's skin 130. Signal processing electronics in the sensor suite extract phasic elements from the sensed data 132. (For example, a band pass filter can restrict measurements with a frequency of 0.2 Hz to 1 Hz, to eliminate outliers.) RMS values of the data are compared to predetermined thresholds 134 and peak values are identified 136.

As shown in FIG. 3, one embodiment includes a physiological monitoring device 200 that can be comfortably applied to the user's skin 10 for long periods of time. The device 200 includes sensing and processing electronics 204 mounted on a clinical-grade medical film 202. The sensing and processing electronics 204 include a wireless communications chipset 206 used to send data to a remote device 210 (which can include a computer, a tablet, a smart phone, etc.). Representations of the data 212 can be displayed for use by a user or a therapist.

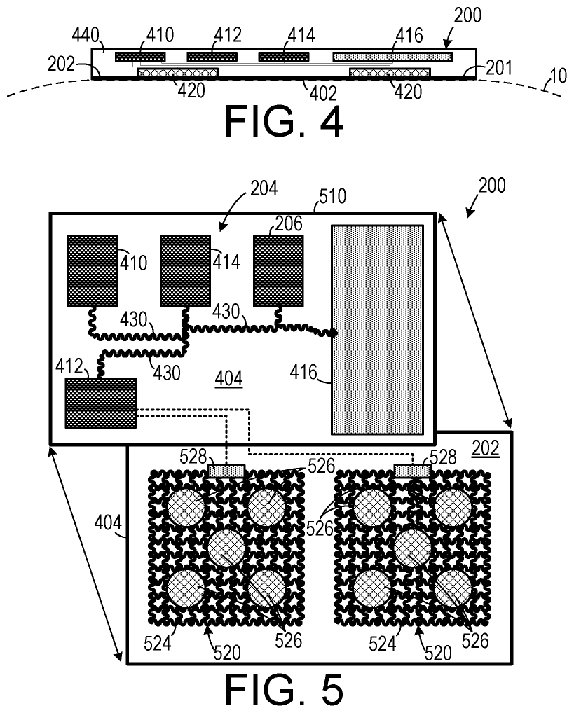

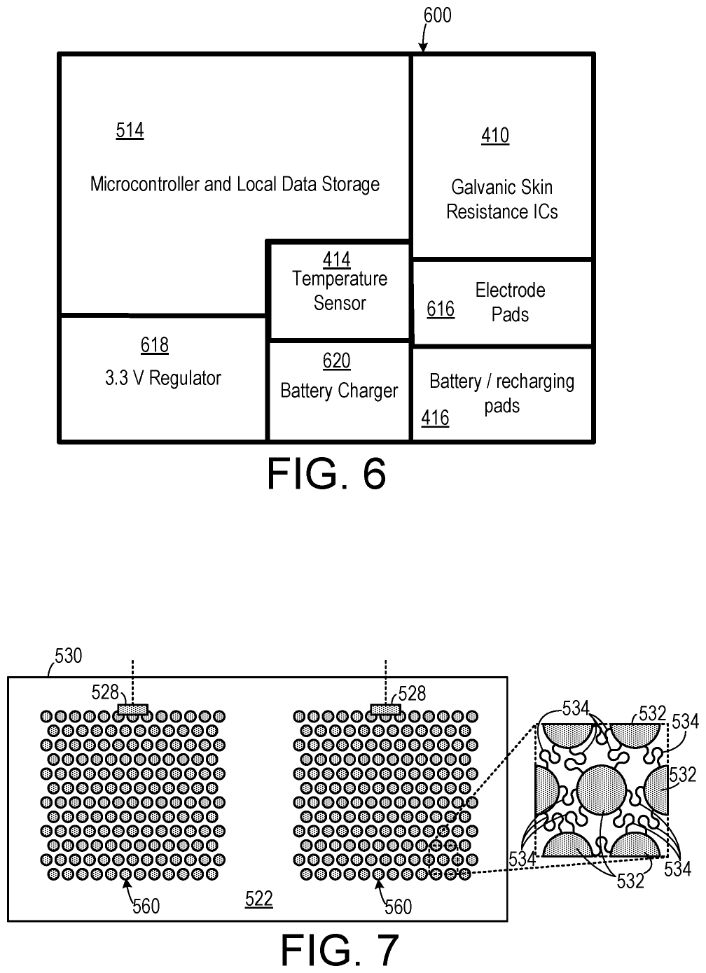

As shown in FIGS. 4-6, the physiological monitoring device 200 has a top side 201 and an opposite bottom side 203 with a skin-compatible adhesive 402 disposed on the bottom side 203. At least two electrodes 420 are disposed on the bottom side 203 and are configured to sense at least one physiological metric (such as GSR, skin temperature, and the like) from the user's skin 10. An elastomeric membrane 404 is integrated with the top side of the clinical-grade medical film 202. A plurality of undulated wires 430 (such as copper wires) is printed on an elastomeric membrane 404. Employing printed undulated wires allows for the monitoring device 200 to be stretched without breaking the wires 430.

The electronic circuit 204 is disposed in the elastomeric membrane 404 and is electrically coupled to the electrodes 420 via the undulated wires 430. The electronic circuit 204 includes a plurality of circuit elements that sense the physiological metrics from the electrodes 420, which convert the physiological metric to a digital value and store the digital value for communication of the digital value to the remote device. The circuit elements can include a microcontroller 514 (including a digital memory), a thermistor 414, a digital potentiometer 410 (for sensing GSR), a wireless communications chipset 206 (e.g., a BlueTooth chipset), a voltage regulator 618, a charging controller 620 and coupling for the electrodes 616. A rechargeable battery 416, which can be recharged either though magnetic contacts or via wireless power transfer, provides power to the circuit elements. An elastomeric envelope 440 (which can include polyimide or polydimethylsiloxane) can surround the electronic circuit 204 and the elastomeric membrane 404.

The electrodes can be formed on electrode pads 520 and include relatively large areas of metal 526 disposed on the membrane 404 that are interconnected via undulated wires 524 also printed on the membrane 404 and connected to connection pads 528. In an alternate arrangement, as shown in FIG. 7, the electrode pads 560 can include an array of relatively small metal areas 532 that are interconnected by undulated wires 534.

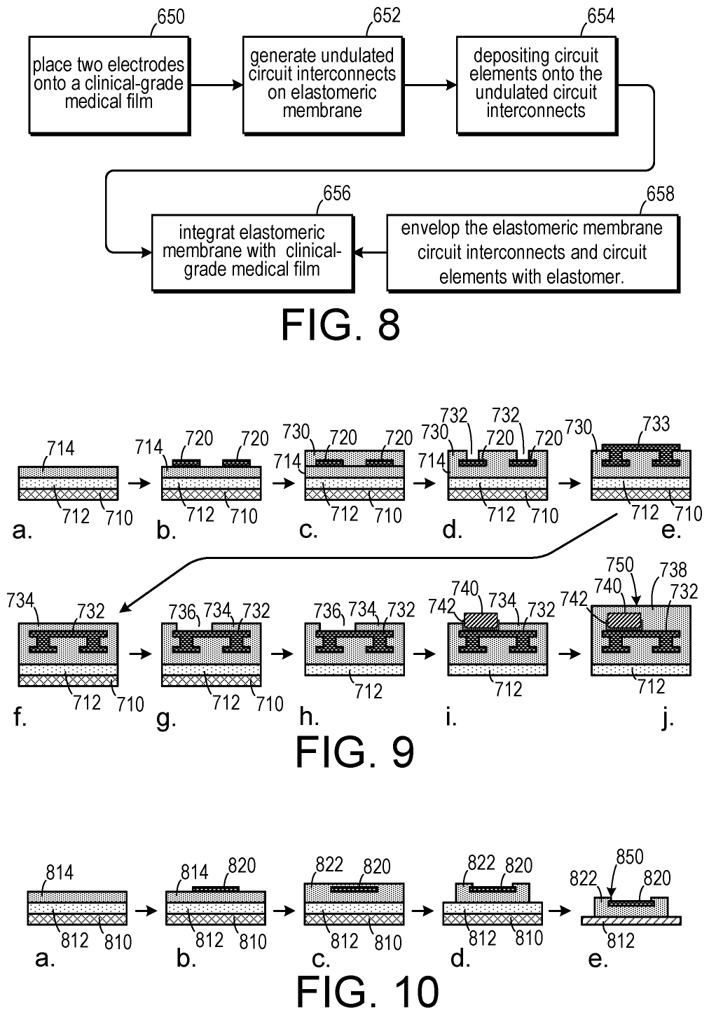

As shown in FIG. 8, in one method of making a skin-mountable electronic circuit, at least two electrodes are deposited 650 onto a clinical-grade medical film having a bottom side with a skin-compatible adhesive so that the at least two electrodes are exposed from the bottom side. A plurality of undulated circuit interconnects is generated 652 on an elastomeric membrane. A plurality of circuit elements is deposited 654 onto the undulated circuit interconnects. The elastomeric membrane is integrated 656 with the clinical-grade medical film so that the electrodes are in electrical communication with the circuit elements and the elastomeric membrane, and the undulated circuit interconnects and the circuit elements are enveloped 658 with an elastomer. When depositing the undulated circuit interconnects, a metallized surface is applied to a wafer, such as a silicon wafer. A photolithography process is employed to define the undulated circuit interconnects on the metallized surface and the undulated circuit interconnects from the wafer are transfer printed to the elastomeric membrane. A rechargeable battery is deposited onto the undulated circuit interconnects prior to the integrating step. The depositing of the electrodes onto a clinical-grade medical film includes sputtering a first conductive metal layer onto a silicon wafer; patterning the first conductive metal to form at least two electrode pads; spin coating at least one elastomer layer on the electrode pads; sputtering a second conductive metal layer onto the at least one elastomer layer; patterning the second conductive layer to form at least two serpentine mesh rectangles; electrically coupling the serpentine mesh rectangles to the electrode pads; and electrically coupling the undulated circuit interconnects to the serpentine mesh rectangles.

In one experimental embodiment, as shown in FIG. 9, the fabrication of the circuit portion employs the following procedure:
Step a.—Spin-coat PDMS 712 (4:1 base-curing-agent ratio) on a Si wafer 700 at 4000 RPM for 30 s, oxygen plasma treatment on PDMS surface for 8 s, spin-coat 1^{st} polyimide layer 714 (PI, PI-2610, HD MicroSystems) at 2000 RPM for 60 s (4.3 μm thickness) and then soft bake at 100° C. for 5 min and hard bake at 250° C. for 1 h.
Step b.—Deposit 0.5 μm thickness of Cu 720 by sputtering.
Step c.—Spin-coat photoresist (PR, Microposit SC1813, MicroChem) at 3000 RPM for 30 s, and soft bake at 100° C. for 5 min. Align with a photomask and expose UV light with intensity of 15 mJ/cm² for 12 s and develop with a developer (MF-319, MicroChem). Etch to form Cu with Cu etchant (APS-100, Transene) and remove remaining PR with acetone, rinse with IPA and DI water. Spin-coat 2^{nd} PI layer 730 (PI-2545, HD MicroSystems) at 2000 RPM for 60 s (3 μm thickness), and soft bake at 100° C. for 5 min. Hard bake at 240° C. for 1 h in a vacuum oven. Spin-coat PR (AZ P4620, Integrated Micro Materials) at 2000 RPM for 30 sec, and soft bake at 90° C. for 4 min. Photolithography exposing UV light with intensity of 15 mJ/cm² for 100 s. Develop with a developer (AZ-400K, Integrated Micro Materials) diluted with DI water (AZ-400K: DI water=1:4).
Step d.—Etch for via hole 732 with reactive ion etcher (RIE) at 250 W, 150 mTorr, and 20 sccm of oxygen for 15 min. Rinse with acetone, IPA, and DI water.
Step e.—Deposit 1.7 μm thickness of 2^{nd} Cu 733 by sputtering.
Step f.—Spin-coat PR (AZ P4620) at 1500 RPM for 30 s, and soft bake at 90° C. for 4 min.
Step g.—Photolithography exposing UV light with intensity of 15 mJ/cm² for 120 s and develop. Etch exposed Cu with Cu etchant. Remove PR with acetone, and rinse with IPA and DI water. Spin-coat 3^{rd} PI layer (PI-2610) at 3000 RPM for 60 s (2.7 μm thickness). Soft bake at 100° C. for 5 min and hard bake at 240° C. for 1 h in a vacuum oven. Spin-coat PR (AZ P4620) at 900 RPM for 30 sec, and soft bakes at 90° C. for 4 min. Photolithography exposing UV light with intensity of 15 mJ/cm² for 160 s and develop. Etch exposed PI with RIE at 250 W, 150 mTorr, and 20 sccm of oxygen for 30 min. Remove remaining PR with acetone, and rinse with IPA and DI water.
Step h.—Peel off the microfabricated circuit with a water-soluble tape (ASWT-2, Aquasol) from the PDMS/Si wafer and put on the 1 mm thickness of silicone elastomer (1:2 mixture of Ecoflex 00-30 and Gels, Smooth-On). Wash the tape with DI water.
Step i.—Mount microchip components 740 with screen-print low-temperature solder paste 742 (alloy of Sn/Bi/Ag (42%/57.6%/0.4%), ChipQuik Inc.). Bake solder at 170° C. for 2 min.
Step j.—Envelop circuit with elastomer to generate circuit portion 750. Download firmware and flash a device through program line connected to circuit with magnetic cubes.

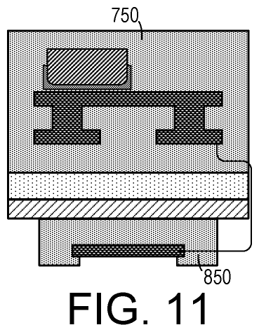

In one experimental embodiment, as shown in FIG. 10, the fabrication of the electrode portion follows the following procedure:
Step a.—Spin-coat PDMS 812 (4:1 base-curing-agent ratio) on a Si wafer 810 at 4000 RPM for 30 s. Oxygen plasma treatment on PDMS surface for 8 s. 1^{st}. Spin-coat polyimide layer 814 (PI-2610) at 2000 RPM for 60 s (4.3 μm thickness). Soft bake at 100° C. for 5 min and hard bake at 250° C. for 1 h.
Step b.—Deposit Cr/Au 820 by sputtering ( 5/200 nm thickness). Spin-coat PR (SC1813) at 3000 RPM for 30 s, and soft bake at 100° C. for 3 min. Photolithography exposing UV light with intensity of 15 mJ/cm² for 12 s and develop with a developer (MF-319). Etch Cr/Au by etchant (Chrome Mask Etchant 9030 and GE-8110, Transene) and remove remaining PR with acetone, rinse with IPA and DI water.
Step c.—Spin-coat 2nd PI layer 822 (PI-2545) at 2000 RPM for 60 s (3 μm thickness), and soft bake at 100° C. for 5 min. Hard bake at 240° C. for 3 h in a vacuum oven. Spin-coat PR (AZ P4620) at 2000 RPM for 30 sec, and soft bakes at 90° C. for 4 min.
Step d.—Photolithography with exposing UV light with intensity of 15 mJ/cm² for 100 s. Develop with a developer. Etch exposed PI except protection layer with RIE at 250 W, 150 mTorr, and 20 sccm of oxygen for 15 min. Remove remaining PR with acetone and rinse with IPA and DI water.
Step e.—Peel off the microfabricated electrodes 850 from the PDMS/Si wafer with a water-soluble tape and put on a medical patch (Tegaderm, 3M, <1 mm thickness). Wash the tape with DI water.

As shown in FIG. 11, the electrodes of the electrode portion 850 are connected to the Cu pads of the circuit portion 750 through a flexible conductive film (ACF, 3M) attached by a silver paste.

In the experimental embodiment, nanomembrane electrodes and stretchable circuits were fabricated using the combination of standard microfabrication, material transfer printing, and soft material packaging. For the electrodes, Cr and Au were sputtered on a Si wafer and patterned into an open-mesh, meander structure, followed by etching steps. For the stretchable circuit fabrication, polyimide and polydimethylsiloxane (PI/PDMS) layers were spin-coated on a Si wafer. Then, the 1st Cu layer was deposited by sputtering and patterned into a serpentine mesh network. Additional layers (PI/Cu/PI) were deposited, and the 1st and the 2nd Cu layers were connected. Then, the circuit surface was etched by reactive ion etcher, leaving PI-insulated Cu traces and exposed Cu pads for subsequent soldering of electronic chip components.

The fabrication of a microstructured circuit on a Si wafer follows the standard micromachining techniques with photolithography, metallization, and etching. The experimental embodiment used an open-mesh, meander (undulated) design to construct the circuit interconnects for mechanical flexibility and stretchability when mounted on the skin. A water-soluble tape facilitated the retrieval of the fabricated circuit patterns for transfer printing onto an elastomeric membrane. A follow-up integration of functional chips (microcontroller, analog front end, thermistor, digital potentiometer, and charging controller) completed the circuit fabrication.

The next step is to assemble the stretchable Au electrodes and circuits with a clinical-grade medical film (e.g., Tegaderm, 3 M, 7×6 cm2). The electrodes are attached to the film's adhesive side while the circuit is placed on top of the film, facilitated by a gel elastomer (1:2 mixture of Ecoflex 00-30 and Gels, Smooth-On). A pair of GSR electrodes have 200 nm in thickness, with the pattern size 2×2 cm2 and inter-distance of 0.3 cm between the pair. The circuit size is 3.1×2.2 cm2, with 2 mm in thickness. The total thickness of the system, including a rechargeable battery, is less than 5 mm.

For continuous monitoring of stress, the system was mounted on the inner wrist. The mechanical characteristics of the compliant circuit and electrodes, can endure stretching, bending, and compression without mechanical failure. The open-mesh structures, used in both electrodes and circuits, accommodate applied strains from bending and stretching.

The system includes an all-in-one, wireless, soft bioelectronic system for portable, continuous monitoring of stress and management practice in daily life. The fully integrated stretchable system incorporates skinconformal nanomembrane electrodes and wireless circuits. The wearable device on the wrist measures high-fidelity GSR and temperature with minimized motion artifacts and enhanced breathability. Simultaneous skin temperature recording provides accurate detection of stress by removing unwanted contributions of temperature changes from sweating. In vivo demonstration with human subjects captures the device performance of continuous stress detection over 6 h during multiple daily activities, including desk work, cleaning, and stress alleviation. Collectively, the bioelectronic stress monitor provides a wearable platform for users to monitor daily stress factors actively and control them via management practice.

For stress monitoring, a fabricated system was mounted on a subject's non-dominant hand's inner wrist. The system measures GSR as the Wheatstone bridge's potential variation. A digital potentiometer actively moves a baseline potential to the central position in the detection range (0-1 V) to increase signal sensitivity. The sampling rate was varied from 1 to 5 Hz, depending on the target recording time. The phasic components of GSR signals were extracted from raw data using the band-pass filter (0.2-1 Hz). The root-mean-square (RMS) value of the phasic components was calculated as a threshold level to detect GSR peaks. The peaks above the threshold were defined as stress arousals and those numbers were counted every minute. Signal-to-noise ratio (SNR) was calculated based on the phasic GSR and noise signals, including motion artifacts identified by the high-pass filter (1 Hz). After RMS conversion of the signals, SNR values (unit: dB) are calculated by the following equation:

$$SNR = 10 {Log}_{10} ( \frac{RMS\_signal}{RMS\_noise} )$$

The all-in-one, low-profile system offers portable, continuous monitoring of stress. Subjects were asked to wear the device on the inner wrist with multiple activities, such as deskwork (reading articles and data analysis) and vacuum cleaning. In addition, each subject attempted stress alleviation activities, such as mindfulness or meditation. For mindfulness, each subject was asked to maintain any restful activity for 10 min. For meditation, each subject was asked to turn on calm music, close the eyes, and keep their mind peaceful while concentrating on breathing for 10 min. The experimental embodiment used a small lithium polymer battery (110 mAh) for the continuous data recording, integrated with the wearable device. This battery could record both GSR and temperature up to 7 h. Afterward, a magnet-assisted connection was required to recharge the battery.

Although specific advantages have been enumerated above, various embodiments may include some, none, or all of the enumerated advantages. Other technical advantages may become readily apparent to one of ordinary skill in the art after review of the following figures and description. It is understood that, although exemplary embodiments are illustrated in the figures and described below, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. It is intended that the claims and claim elements recited below do not invoke 35 U.S.C. § 112 (f) unless the words “means for” or “step for” are explicitly used in the particular claim. The above-described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A physiological monitoring device for application to a user's skin, comprising:
(a) a clinical-grade medical film having a top side and an opposite bottom side with a skin-compatible adhesive disposed on the bottom side;
(b) at least two electrodes disposed on the bottom side and configured to sense at least one physiological metric from the user's skin;
(c) an elastomeric membrane that is integrated with the top side of the clinical-grade medical film;
(d) a plurality of undulated wires printed on an elastomeric membrane; and
(e) an electronic circuit disposed on the elastomeric membrane and electrically coupled to the at least two electrodes via the plurality of undulated wires, the electronic circuit including a plurality of circuit elements that sense the at least one physiological metric from the at least two electrodes, that convert the at least one physiological metric to a digital value, and that store the digital value for communication of the digital value to a remote device.

2. The physiological monitoring device of claim 1, wherein the at least one physiological metric comprises galvanic skin resistance.

3. The physiological monitoring device of claim 1, wherein the at least one physiological metric comprises skin temperature.

4. The physiological monitoring device of claim 1, wherein the plurality of circuit elements comprises: a microcontroller, a thermistor, a digital potentiometer and a charging controller.

5. The physiological monitoring device of claim 1, further comprising a rechargeable battery that provides power to the plurality of circuit elements.

6. The physiological monitoring device of claim 1, further comprising an elastomeric envelope surrounding the electronic circuit and the elastomeric membrane.

7. The physiological monitoring device of claim 6, wherein the elastomeric envelope comprises at least one of polyimide and polydimethylsiloxane.

8. The physiological monitoring device of claim 1 wherein the undulated wires comprise copper.

9. A method of making a skin-mountable electronic circuit, comprising the steps of:
(a) depositing at least two electrodes onto a clinical-grade medical film having a bottom side with a skin-compatible adhesive so that the at least two electrodes are exposed from the bottom side;
(b) generating a plurality of undulated circuit interconnects on an elastomeric membrane;
(c) depositing a plurality of circuit elements onto the undulated circuit interconnects;
(d) integrating the elastomeric membrane with the clinical-grade medical film so that the electrodes are in electrical communication with the circuit elements; and
(e) enveloping the elastomeric membrane, the undulated circuit interconnects and the circuit elements with an elastomer.

10. The method of claim 9, wherein the step of depositing a plurality of undulated circuit interconnects comprises the steps of:
(a) applying a metallized surface to a wafer;
(b) employing a photolithography process to define the undulated circuit interconnects on the metallized surface; and
(c) transfer printing the undulated circuit interconnects from the wafer to the elastomeric membrane.

11. The method of claim 10, wherein the wafer comprises a silicon wafer.

12. The method of claim 9, further comprising the step of depositing a rechargeable battery onto the undulated circuit interconnects prior to the integrating step.

13. The method of claim 9, wherein the plurality of circuit elements comprises: a microcontroller, a thermistor, a digital potentiometer and a charging controller.

14. The method of claim 9, wherein the step of depositing at least two electrodes onto a clinical-grade medical film comprises the steps of:
(a) sputtering a first conductive metal layer onto a silicon wafer;
(b) patterning the first conductive metal to form at least two electrode pads;
(c) spin coating at least one elastomer layer on the electrode pads;
(d) sputtering a second conductive metal layer onto the at least one elastomer layer;
(e) patterning the second conductive layer to form at least two serpentine mesh network rectangles;
(f) electrically coupling the at least two serpentine mesh rectangles to the at least two electrode pads; and
(g) electrically coupling the undulated circuit interconnects to the serpentine mesh rectangles.

15. The method of claim 14, wherein the at least one elastomer comprises at least one of polyimide and polydimethylsiloxane.

16. The method of claim 9, further comprising the step wherein the undulated circuit interconnects comprise copper.

17. A method of monitoring a stress level of a user, comprising the steps of:
(a) applying a circuit for sensing at least one physiological metric that is mounted on a substrate that includes a flexible, stretchable and breathable material to the skin of the user;
(b) sensing the at least one physiological metric and storing a digital value representative thereof in the circuit;
(c) reading the digital value representative of the at least one physiological metric with a remote device;
(d) correlating a digital value representative of the at least one physiological metric to a corresponding stress level; and
(e) presenting data to the user indicative of the stress level.

18. The method of claim 17, wherein the substrate comprises a clinical-grade medical film and an elastomeric membrane.

19. The method of claim 17, wherein the at least one physiological metric comprises galvanic skin resistance.

20. The method of claim 17, wherein the at least one physiological metric comprises temperature.
